(19) United States
(12) Patent Application Publication
(10) Pub. No.: US 2020/0361842 A1
(43) Pub. Date: Nov. 19, 2020

(54) Preparation and Characterization of Cardanol Based Vinyl Ester Resins as Cross-linker Units

(71) Applicants: DREXEL UNIVERSITY, Philadelphia, PA (US); The Government of the United States of America, as represented by The Secretary of the Army, Washington, DC (US)

(72) Inventors: Giuseppe R. Palmese, Hainesport, NJ (US); Emre Kinaci, Philadelphia, PA (US); John J. LaScala, Wilmington, DE (US)

(73) Assignees: Drexel University, Philadelphia, PA (US); The Government of the United States of America, as represented by The Secretary of the Army, Washington, DC (US)

(21) Appl. No.: 16/758,719

(22) PCT Filed: Oct. 23, 2018
(86) PCT No.: PCT/US2018/057048

§ 371 Date: Apr. 23, 2020

Related U.S. Application Data
(60) Provisional application No. 62/576,368, filed on Oct. 24, 2017.

(51) Int. Cl. C07C 39/19; C08F 222/20

(52) U.S. Cl. CPC C07C 39/19; C08F 222/20

(57) ABSTRACT

A polymerizable monomer prepared by partial or complete acrylation or methacrylation of at least one epoxidized cardanol, wherein the epoxidized cardanol is formed by epoxidation of an unsaturation site of a cardanol having the formula (I) wherein R is selected from hydrogen and an alkyl, or alkenyl group having 1-6 carbon atoms, and n is 7, 10, or 13, a polymer and resin made form the polymerizable monomer, and a method of preparing such a polymer. The resin created from this monomer is suitable for moderate temperture composites and coatings applications.

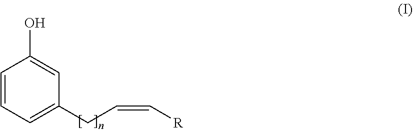

### CROSS REFERENCE TO RELATED APPLICATIONS

This application claims the benefit of U.S. Provisional Application No. 62/576,368, filed Oct. 24, 2017, the entire disclosure of which is hereby incorporated by reference as if set forth fully herein.

### STATEMENT OF GOVERNMENT INTEREST

This invention was made with government support under Contract Number W911NF-13-2-0046 awarded by the United States Army Research Laboratory. The Government has certain rights in this invention.

### FIELD OF THE INVENTION

The present invention relates to polymerizable monomers prepared by partial or complete acrylation or methacrylation of at least one epoxidized cardanol, polymers and resins formed from the polymerizable monomer and methods of forming the same.

### BACKGROUND

Vinyl ester resins (VERs) are the addition products of various epoxide resins and unsaturated monocarboxylic acids, such as methacrylic acid. These reactive vinyl groups can homopolymerize to form a cross-linked network or they can be cured with a vinyl type reactive diluent such as styrene that takes part in the reaction. Because of their high viscosity, it is common to dilute VERs with a low molecular weight mono-vinyl co-monomer, most commonly styrene, to reduce the viscosity of the resin to processable range which is typically between 200-2000 cPs. VERs combine good thermal and mechanical properties of the epoxy resins with the rapid and low temperature curing nature of the unsaturated polyester resins. These resins are extensively used as surface coatings, adhesives, molding compounds, structural laminates, composites and electrical insulation products because of their good mechanical and thermal properties and chemical resistance.

Commercially available VERs are generally prepared by the reaction between Diglycidyl Ether of Bisphenol A (DGEBA) and methacrylic acid. Due to the high viscosity of the VERs, the modified resin is dissolved in 30-60 wt. % reactive diluent such as styrene to achieve the low viscosity of a liquid molding resins. Among the reactive diluents, styrene is the most common one because of its low cost and high reactivity while it imparts good mechanical properties and heat distortion. However, styrene and other commonly used reactive diluents with VERs are hazardous air pollutants (HAP) and volatile organic compounds (VOCs) so there has been an effort to replace these monomers with less volatile alternative one. Furthermore, the most commonly used vinyl ester resins are based on DGEBA which is derived from bisphenol A (BPA). BPA is classified as a reprotoxic R2 substance. This endocrine disruptor can mimic the body's own hormones and may lead to several negative health effects including the alterations in both the immune and reproductive systems along with a modification in brain chemistry. The negative effect of BPA and other synthetic based monomers on human health and environment necessary implies focusing research on the substitution of BPA in epoxy and vinyl formulations as well as the synthetic reactive diluents which are HAP and VOC with non-toxic alternatives. Replacing these synthetic based reactive diluents, such as styrene, with plant derived ones not only contributes to global sustainability, but they may result in improved properties, such as fire retardancy and improved fracture toughness. Thus, replacing BPA based DGEBA and volatile, petroleum-based styrene with the nature derived ones is an important and urgent subject in academia as well as industry because the US government has serious regulations for the use of these volatile diluents in thermosetting formulations.

Among the plant-based materials that can be used in the synthesis of thermosetting materials, Cashew Nut Shell Liquid (CNSL) is an important one due to its low price, high availability, unique structure and reactive functionalities. CNSL is a viscous reddish-brown liquid which can be extracted from the outer shells of the cashew nut fruit. It is a non-edible oil and it is native to tropical or subtropical countries such as India and Brazil. Cardanol which is the main component of thermally treated CNSL is a phenol derivative having a C15 alkyd chain at the meta position. The C15 alkyd chain of this phenol derivative has varying degrees of unsaturation: 8.4% saturated, 48.5% mono-unsaturated, 16.8% di-unsaturated, and 29.3% tri-unsaturated with the double bonds, respectively in positions (8), (8, 11), and (8, 11, 14). Due to cardanol's unique structure and functionality, it has been used for many applications such as pre-polymers, paint and primers, plasticizers etc. In addition, Cardanol's combination of a rigid, chemically resistant phenol ring with a hydrophobic tail makes this material good candidate for corrosion-resistant coating applications.

So far, a few studies have concentrated on synthesis and utilization of cardanol based vinyl ester in thermoset synthesis. Because of its reactive functionalities, cardanol molecule can be used as a cross-linker unit as well as a diluent in VE synthesis. Significant amount of studies has been published on the synthesis of cardanol based epoxidized novolac resin via reaction with methacrylic acid and its curing kinetics with and without styrene as a reactive diluent. Although this resin had low viscosity and high reactivity, the presence of the uncrosslinked C15 side chain in the polymer backbone resulted in a significant reduction in thermo-mechanical and mechanical properties with respect to BPA based VERs. In addition, synthesis of this cardanol based novolac resin required a three step reaction and use of volatile solvents such as epichlorohydrin and formaldehyde which reduces the sustainability of the material.

A related study, as described by Jaillet, F.; Nouailhas, H.; Auvergne, R.; Ratsimihety, A.; Boutevin, B.; Caillol, S. European Journal of Lipid Science and Technology 2014, 116, (7), 928-939, where cardanol based VER was utilized as a cross-linker unit in formulations, the cardanol based di-phenyl di-epoxy (NC514) and petroleum based di epoxy DGEBA resins were methacrylated in the presence of triphenylphosphine (TPP) catalyst (1 wt. %) and hydroquinone inhibitor (0.25 wt. %). The resultant methacrylate analogs of NC514 and DGEBA were mixed with several reactive diluents at 40 wt % diluent ratio. Thermal degradation profiles of the fully cured NC514 and DGEBA based formulations did not show a significant difference as determined via TGA studies; however, the tan delta glass transition temperature of the NC514 based formulations were significantly lower than the DGEBA based formulations as demonstrated via DMA studies.

Previously, cardanol based VERs as a cross-linker as well as a reactive diluent in vinyl ester formulations have been utilized to create full and partially cardanol based VE networks, as described in Can, E.; Kinaci, E.; Palmese, G. R. European Polymer Journal 2015, 72, 129-147. Cardanol based NC514 and synthetic DGEBA were subjected to esterification reaction along with mono-epoxy cardanol molecule (LITE 2513HP). The methacrylated analog of cardanol based NC514 (NC514VE) was mixed with styrene and methacrylated analog of mono-epoxy cardanol (LITEVE) at different weight ratios ranging from 10-50 wt. % diluent comonomer content. For comparison, methacrylated version of DGEBA (DGEBAVE) was also mixed with cardanol based LITEVE co-monomer at listed concentrations. The thermo-mechanical and mechanical characterization of the NC514 and DGEBA based formulations showed significantly lower Young's Modulus and loss modulus glass transition temperatures for NC514-based polymers as expected because of the C15 alkyd chain that reduces the stiffness and thermal stability of the polymer. In addition, using cardanol based diluent in VE formulations reduced these properties significantly with respect to using styrene as a diluent co-monomer.

What is needed is a cardanol-based building block that has the properties sufficient to replace BPA for composite and corrosion prevention applications.

### SUMMARY OF THE INVENTION

In accordance with the disclosure, exemplary embodiments provide polymerizable monomers prepared by partial or complete acrylation or methacrylation of at least one epoxidized cardanol, a polymer and resin formed from the polymerizable monomer and methods of forming the same.

The following are sentences describing embodiments of the invention.

1. A polymerizable monomer prepared by partial or complete acrylation or methacrylation of at least one epoxidized cardanol, wherein the epoxidized cardanol is formed by epoxidation of an unsaturation site of a cardanol having the formula:

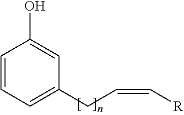

wherein R is selected from hydrogen and an alkyl, or alkenyl group having 1-6 carbon atoms, and n is 7, 10, or 13.
2. The polymerizable monomer of sentence 1, wherein said epoxidized cardanol is cardanol glycidyl ether.
3. The polymerizable monomer of any one of sentences 1-2, prepared by reacting (meth)acrylic acid with the at least one epoxidized cardanol in the presence of a metal-containing catalyst.
4. The polymerizable monomer of any one of sentences 1-2, prepared by reacting (meth)acrylic anhydride with the at least one epoxidized cardanol.
5. The polymerizable monomer of any one of sentences 1-2, prepared by reacting (meth)acryloyl chloride with the at least one epoxidized cardanol in the presence of one or or more of triethylamine, tertiary amines, pyridines and pyridine derivatives.
6. The polymerizable monomer of any one of sentences 3-5, wherein the at least one epoxidized cardanol has from about 2.0 to about 4.0 epoxy groups per molecule.
7. The polymerizable monomer of any one of sentences 3-5, wherein the at least one epoxidized cardanol has from about 2.2 to about 3.0 epoxy groups per molecule.
8. The polymerizable monomer of any one of sentences 1-7, wherein the (meth)acrylation is carried out in the presence of an effective amount of a free radical polymerization inhibitor.
9. The polymerizable monomer of any one of sentences 1-8, wherein the (meth)acrylation is carried out in the presence of an effective amount of a catalyst for carboxylic acid-epoxy resin systems, said catalyst being selected from of amines, phosphines, antimony compounds, and imidazoles.
10. The polymerizable monomer of any one of sentences 1-9, wherein the (meth)acrylation is carried out using a molar ratio of (meth)acrylate groups to epoxide groups in the epoxidized cardanol of from about 0.5 to about 1.1.
11. A polymerizable monomer prepared by partial or complete acrylation or methacrylation of at least one hydroxylated cardanol, wherein the hydroxylated cardanol is formed by hydroxylation of at least one unsaturation site of a cardanol having the formula:

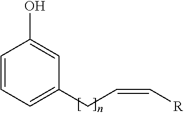

wherein R is selected from hydrogen, and an alkyl group, or alkenyl goup having 1-6 carbon atoms, and n is 7, 10, or 13.
12. The polymerizable monomer of sentence 11, prepared by reacting (meth)acrylic anhydride with the at least one hydroxylated cardanol.
13. The polymerizable monomer of sentence 11, prepared by reacting (meth)acryloyl chloride with the at least one hydroxylated cardanol in the presence of triethylaluminium.
14. The polymerizable monomer of any one of sentences 11-13, wherein the (meth)acrylation is carried out using a molar ratio of (meth)acrylate groups to hydroxy groups in the hydroxylated cardanol of from about 0.5 to about 1.1.
15. A polymerizable monomer comprising a vinyl ester of the formula:

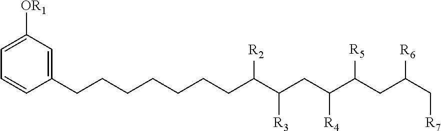

wherein R₁ is selected from one of the following, hydrogen, propylene oxide,

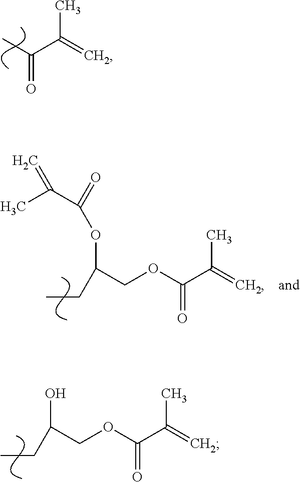

and
R₂, R₃, R₄, R₅, R₆ and R₇ are independently selected from hydrogen, hydroxyl, an acrylate group, a methacrylate group, and an epoxy groups formed by combinations of R₂ and R₃, R₄ and R₅, and R₆ and R₇; and wherein at least one of R₂, R₃, R₄, R₅, R₆ and R₇ includes an acrylate group or a methacrylate group.
16. A method of making a polymer comprising the step of curing a composition comprising the polymerizable monomer of any one of sentences 1-14.
17. The method of sentence 16, wherein the composition further comprises from 1 to 99% by weight of at least one reactive diluent, based on the total weight of the composition.
18. The method of sentence 17, wherein the reactive diluent is selected from the group consisting of: styrene, 2-hydroxymethacrylate, methyl methacrylate, methyl acrylate, furfuryl methacrylate, methacrylated lauric acid, methacrylated C₆-C₂₄ fatty acids, methacrylated guaiacol, methacrylated phenol, and methacrylated eugenol.
19. The method of any one of sentences 17-18, wherein the reactive diluent is a methacrylated C₆-C₂₄ fatty acid.
20. The method of any one of sentences 17-19, wherein said composition comprises from about 50% to about 90% by weight of the polymerizable monomer of any one of claims 1-14 and from about 10% to about 50% by weight of the reactive diluent, based on a total weight of the composition.
21. The method of any one of sentences 17-19, wherein the composition comprises from about 30% to about 50% by weight of said reactive diluent.
22. The method of any one of sentences 17-21, wherein the curing step is carried out in the presence of an effective amount of a polymerization initiator.
23. A polymer obtained by the method of any one of sentences 17-22.
24. The polymer of sentence 23 having a Tg of from about 0° C. to about 125° C., wherein the Tg is the temperature at which the largest peak of the loss modulus occurs, as measured by Dynamic Mechanical Analysis method IOP L1047-033 with the following parameters: test frequency=1 Hz, strain=0.02%, and heating rate of 2° C./min, a modulus of more than 1 GPa at 25° C. and a strength of more than 10 MPa at 25° C.
25. The polymer of sentence 23, having a Tg of from about 100° C. to about 150° C. wherein the Tg is the temperature at which the largest peak of the loss modulus occurs, as measured by Dynamic Mechanical Analysis method IOP L1047-033 with the following parameters: test frequency=1 Hz, strain=0.02%, and heating rate of 2° C./min.
26. A resin comprising the polymerizable monomer of any one of sentences 1-14 and a reactive diluent, wherein the resin has a viscosity of from about 500 cP to about 2000 cP.

### BRIEF DESCRIPTION OF THE DRAWINGS

FIG. 1 shows cross-linkers and reactive diluents used in the examples

FIG. 2 shows the structure verification of the SCECGEVE via ¹H-NMR

FIG. 3 shows viscosity measurements of different VE formulations as a function of reactive diluent content at 30° C. and a 25 s⁻¹ shear rate.

FIGS. 4A-4B show the mid-IR spectrum of the DGEBAVE and SCECGEVE samples before cure (bottom) and after post-cure (top) in the presence of 30 wt. % ST (FIG. 4A) and 30 wt. % MFA (FIG. 4B).

FIG. 5 shows the extent of cure as indicated by vinyl bond conversion as a function of ST and MFA content for the SCECGEVE cross-linker.

FIGS. 6A-6B show the temperature dependence of loss and storage modulus for different cross-linker units at 30 wt. % ST content (FIG. 6A) and 30 wt. % MFA content (FIG. 6B).

FIGS. 7A-7B show the temperature dependence of loss and storage modulus for SCECGEVE resin as a function of ST content (FIG. 7A) and MFA content (FIG. 7B).

FIGS. 8A-8B show the temperature dependence tan delta for SCECGEVE resin as a function of ST content (FIG. 8A) and MFA content (FIG. 8B) along with neat cross-linker SCECGEVE.

FIG. 9 shows a comparison of tan delta Tg values as obtained from DMA measurements and from Fox Analysis as a function of diluent content. The Dashed Line: Predicted via Flory Fox equation

FIGS. 10A-10B show thermal degradation profiles of fully cured samples of different cross-linker units at 30 wt. % (FIG. 10a) ST content (FIG. 10b) MFA content.

### DETAILED DESCRIPTION OF THE EXEMPLARY EMBODIMENTS

Disclosed herein are polymerizable monomers prepared by partial or complete acrylation or methacrylation of at least one epoxidized cardanol.

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other systems and methods. Before explaining the disclosed embodiments of the present invention in detail, it is to be understood that the invention is not limited in its application to the details of any particular embodiment shown. Additionally, the terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

As used herein and in the appended claims, the singular forms “a”, “an”, and “the” include plural references unless the context clearly dictates otherwise. The terms “a” (or “an”), “one or more” and “at least one” can be used interchangeably herein. It is also to be noted that the terms “comprising”, “including”, and “having” can be used interchangeably.

As used herein “room temperature” refers to a temperature of 18° C.

Epoxy Groups with (Meth)acrylic Acid

(Meth)acrylated epoxy cardanol can be prepared using the following method. Cardanol is obtained and epoxidized through known unsaturation epoxidation methods. The unsaturation epoxidation may, in certain embodiments, be combined with glycidyl phenyl epoxidation using known methods. The product of the epoxidation step(s) can be converted to a (meth)acrylic ester by reaction with a slight excess of acrylic acid or methacrylic acid at 70-120° C., or, in certain embodiments at 90-100° C., and in other embodiments at 80-90° C., in some cases using a catalyst, such as AMC-2, triphenylphosphine, or triphenylantimony(III). The reaction may be conducted for 1-5 hours, or, in certain embodiments 2-3 hours. Acid number can be used to verify addition of the (meth)acrylic acid. In certain embodiments, an acid number of less than 20 is obtained. NMR analysis can be used to verify that nearly two (meth)acrylate groups are present per molecule.

In another embodiment, partially (meth)acrylated epoxy cardanol can be prepared from epoxidized cardanol by reaction with acrylic acid or methacrylic acid at 70-120° C., or, in certain embodiments at 90-100° C., and in other embodiments at 80-90° C., using a catalyst, such as AMC-2, triphenylphosphine, or triphenylantimony(III). The reaction may be conducted for 1-5 hours, or, in certain embodiments 2-3 hours. To obtain the partially (meth)acrylated epoxy cardanol, the amount of (meth)acrylic acid used is less than the stoichiometric amount for reaction with the epoxy groups of the epoxy cardanol, such as, for example, in certain embodiments 25-75% of the stoichiometric amount. Acid number can be used to verify addition of the (meth)acrylic acid; and in certain embodiments, an acid number of less than 15 is observed. NMR analysis can be used to verify the number of (meth)acrylate groups and epoxide groups present per molecule.

Hydroxyl Groups with (Meth)acryloyl Chloride or (Meth)acrylic Anhydride

Epoxidized cardanol can be functionalized through various alternative methods and converted to produce methacrylated and acrylated phenolics that are capable of free radical polymerization. Although epoxidized cardanol is the preferred starting material for these additional methods, hydroxylated alkyl chains attached to the cardanol can also be employed. The methods of creating the epoxidized cardanol are described herein and are known in the art. The hydroxylated alkyl chains can be added to the cardanol by hydroxylation of the unsaturation sites. This can be done in a manner whereby each unsaturation yields two hydroxyl groups or in a manner whereby each unsaturation yields one hydroxyl group and one alkoxy group. There are standard/well known preparation techniques that can be used to prepare these species.

The partially, or fully (meth)acrylated cardanol is formed by esterification using, for example, (meth)acryloyl chloride or (meth)acrylic anhydride and a base catalyst (for example 4-(dimethylamino)pyridine or triethylamine) in an aprotic solvent (for example dichloromethane or tetrahydrofuran). The anhydrides and acid chlorides will react twice with an epoxy group and will not form hydroxyl groups thereby reducing the viscosity. Reaction temperatures in certain embodiments are 20-80° C., and in other embodiments, 25-55° C. A base catalyst in an aprotic solvent or no solvent may be used. Use of solvent requires distillation to remove the solvent and is thus not desired for manufacturing purposes.

Synthesis can be carried out using a similar methodology utilizing acryloyl chloride or acrylic anhydride as the (trans)esterification agents. The identity of the products can be ascertained by NMR analysis. Use of acid chlorides requires extraction of the HCl that is produced. Triethylamine or pyridine catalysts may be used to sequester the acid. Extraction of the cardanol derivatives in ethyl ether, dichloromethane, or chloroform with washes in water, basic water (typically sodium bicarbonate) and salt water is done to purify the solvent phase.

When hydroxyl groups are remaining on the cardanol molecule, or when hydroxylated cardanol is used, maleic anhydride can be reacted with the hydroxyl groups in a post treatment. Maleic anhydride may be added to the cardanol derivative in amounts up to stoichiometric amounts of maleic anhydride to hydroxyl groups on the cardanol derivative.

The reaction is catalyzed using an acid catalyst, for example p-toluenesulfonic acid, AMBERLYST™ 15 hydrogen form or DOWEX™ DR-2030 hydrogen form, and can be carried out in the presence of various organic solvents, such as toluene and xylenes.

The examples report the synthesis and characterization of a cardanol based low viscosity novel VER derived from Side Chain Epoxidized Cardanol Glycidyl Ether (SCECGE) with approximately 2.45 epoxy/molecule whose synthesis and characterization was reported previously by Can, E.; Kinaci, E.; Palmese, G. R. European Polymer Journal 2015, 72, 129-147. This methacrylated VER was incorporated with petroleum based reactive diluent styrene and plant oil based reactive diluent methacrylated lauric acid (MFA) at 10-40 wt. % diluent content. For comparison the commercially available cardanol based resin NC514 with approximately 2 epoxies/oligomer, and petroleum based DGEBA with 2 epoxies/molecule were methacrylated and mixed with the listed reactive diluents at the same concentrations. Without being bound by theory, it is believed that the additional methacrylate functionality on this monomer increases the thermal and mechanical properties of SCECGEVE relative to that of NC514VE and approaches that of DGEBAVE resins. Unlike epoxy resins based on SCECGE where the 1.45 secondary epoxides per molecule have low reactivity, the methacrylates on SCECGVE have good reactivity.

To assess the processability of these VE resins, the viscosities of the different compositions were measured prior to curing using a rheometer. The extent of curing of the SCECGEVE, NC514VE and DGEBAVE VERs were analyzed via FT-IR after post-curing to assess monomer structure effects on network formation and to enable structure-property evaluation of the resin. The storage modulus and glass transition temperatures (Tg's) of the cured resins were determined via DMA and thermal degradation profiles were explored via TGA studies. Mechanical properties of all the cured VERs were determined via tensile tests.

### EXAMPLES

### 2.1 Materials

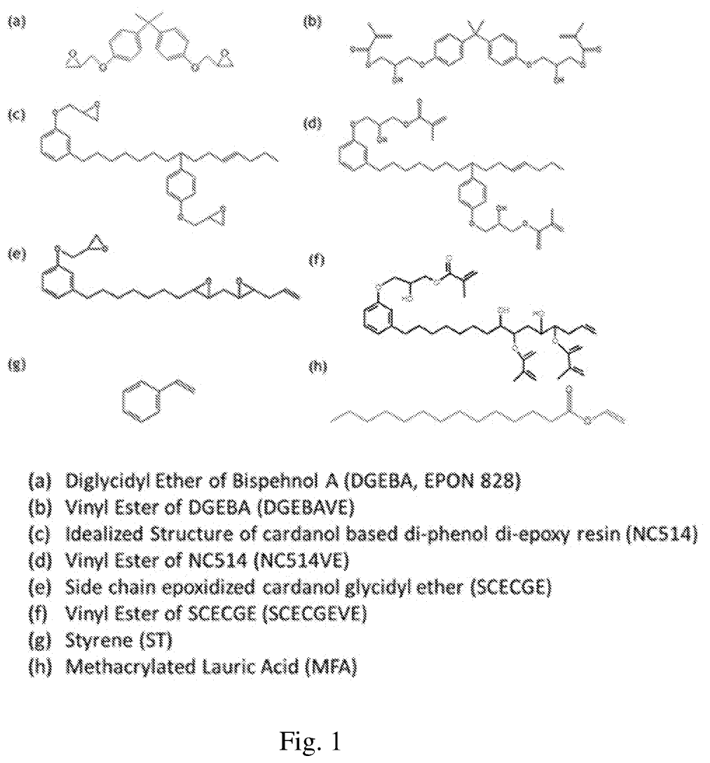

Cardanol based di-phenol di-epoxy resin NC514 (EEW:400 g/eq) was obtained from Cardolite. Diglycidyl ether of bispheol A (DGEBA, EEW:188 g/eq) was obtained from Miller Stephenson (Danbury, Conn.). Methacrylated lauric acid (MFA) was prepared as it was previously reported by Khot, S. N.; Lascala, J. J.; Can, E.; Morye, S. S.; Williams, G. I.; Palmese, G. R.; Kusefoglu, S. H.; Wool, R. P. Journal of Applied Polymer Science 2001, 82, (3), 703-723, and cardanol based Side Chain Epoxidized Cardanol Glycidyl Ether (SCECGE) resin was also prepared as previously reported. All epoxy resins used in this study were kept in a vacuum oven at 80° C. for 1 hour to remove volatile residues and degas the resin prior to the methacrylation reactions. Methacrylic acid (MA) (>99%), styrene (ST), (>99.5%), and hydroquinone (>99.5%) were obtained from Sigma Aldrich (USA) and used as received. AMC-2, which is a mixture of 50% trivalent organic chromium complex and 50% phthalate ester (Aerojet Chemicals, Rancho Cordova, Calif.) was used as a catalyst for methacrylation reaction. Trigonox 239A was obtained from Akzo Nobel Chemicals (Chicago, Ill.) and CoNap which contains 45% cumene hydroperoxide, and cobalt naphthalene were used as the curing catalyst/initiator for the vinyl ester resins (VERs). Structures of the cross-linkers and reactive diluents used in the study are presented in FIG. 1.

### 2.2. Preparation of VE Resins

Vinyl ester resins were prepared via methacrylation of DGEBA, and cardanol based NC514, and SCECGE in the presence of 1.0 wt % AMC-2 catalyst and 0.1 wt % hydroquinone to inhibit the free radical polymerization at elevated temperatures. Prior to the synthesis of the VERs, the epoxy equivalent weights (EEW) of the unmodified resins were determined according to ASTM D1652-11 standard test method. EEW values obtained from these titrations were to determine the amount of methacrylic acid that should be used for the esterification reaction. In a representative procedure, epoxy resins were charged in to a 250 mL round bottom flask together with 1.01 the stoichiometric amount of methacrylic acid, and previously specified amounts of AMC-2 catalyst, and hydroquinone and reacted at 90° C. for 4 hours with constant magnetic stirring. The methacrylated analogs of the epoxy resins were dubbed as DGEBAVE, NC514VE, and SCECGEVE.

### 2.3. Characterization of VE Resins

A Varian Innova NMR unit (500 MHz) was used to confirm the final structures of the synthesized VE monomers. Samples were prepared by dissolving the VERs in CDCl₃ and 32 scans were collected per sample at 298K at 90° pulse width.

Acid number titrations were performed to measure the amount of unreacted methacrylic acid in the VE systems. For this purpose, 1 g of the reaction mixture was dissolved in 5 g acetone. Three to four drops of 0.5 wt % phenolphthalein in 50% ethanol were added to the mixture as the indicator solution. The solution was then titrated with 0.5N sodium hydroxide until the solution remained slightly pink for at least 30 s. An acid number value less than 10 corresponding to ˜3 wt. % free acid was the maximum allowable acid number.¹¹ If the acid number was too high, the reaction was allowed to continue until the acid value dropped below 10.

### 2.4 Preparation of Cured Samples of VERs

The VE formulations were prepared by mixing the cardanol based SCECGEVE with bio-based methacrylated lauric acid (MFA) and petroleum based styrene (ST) at different co-monomer contents (10-40 wt %) to create fully and partially bio-based VE formulations. For comparison, petroleum based DGEBAVE and cardanol based NC514VE was also mixed with listed diluents at 30 wt % content. All the prepared mixtures were first characterized for rheological properties before cure and then 1.5 wt. % Trigonox A and 0.5 wt. % CoNap catalyst-initiator complex were added to the VE mixtures and the contents were mixed and degassed using a high shear mixer (Thinky, AR 2000). After complete mixing was achieved, VE samples with styrene were placed in to preheated metal molds and that were then sealed prevent significant styrene evaporation. Samples with non-volatile MFA were also transferred in to preheated rubber molds. The approximate dimensions of the metal and rubber mold for DMA testing were ˜35×12.5×5 mm³. For tensile testing, dog bone shaped metal molds were used to obtain fully cured samples of VE with styrene and MFA. The molded resins were kept in room temperature for 12 hours and then placed at 120° C. for another 12 hours and another post-curing step was applied at 160° C. for 3-4 hours. After the full cure of the resins, samples were de-molded and polished to make them uniform in dimensions for DMA and tensile testing.

### 2.5 Viscosity Measurements of the VE Resins

The viscosities of the VE cross-linkers and resin mixtures with different reactive comonomer concentrations were measured prior to curing and catalyst/initiator addition. All VE samples tested at 25° C. by using a TA Instrument Rheometer (AR2000 ex) while increasing the shear rate from 0.2 to 200 s⁻¹. VE samples including volatile ST was tested with concentric cylinder geometry while samples containing nonvolatile MFA tested via parallel plate geometry. The values produced by concentric cylinder geometry and parallel plate geometry are compatible to each other.

### 2.6. Extent of Curing Analysis

Mid-IR was used to calculate the extent of cure for the VE resin blends based on the consumption of the methacrylate and styrene vinyl ester groups. VERs containing 30 wt % comonomer, before cure and after post-cure, were scanned in 500-4000 cm⁻¹ range at room temperature with a total of 32 scans per spectrum at 8 cm⁻¹ resolution. SCECGEVE based samples were also evaluated in the presence of 0-70 wt % MFA and ST diluent to consider the effect of comonomer on extent of curing. Liquid, uncured VE blends were drop cast on the ATR diamond and the post cured samples were DMA bars with thickness ranging 2 to 3 mm.

### 2.7. Cured Sample Properties

Dynamic Mechanical Analysis (TA Instruments, Q800) was used to evaluate the cross-link density (ν), glass transition temperature, and the room temperature storage modulus (E′) of the cured polymers. Standards and methods for the tests that were utilized to assess sample properties are described in Joshua Sadler, Ian M McAninch, Faye R Toulan, Felicia Levine, and John J La Scala, Resin Characterization, ARL-SR0323, June 2015. Specifically, Dynamic Mechanical Analysis followed method IOP L1047-033 with the following parameters: test frequency=1 Hz, strain=0.02%, and heating rate of 2° C./min. To more accurately measure the glassy behavior, rectangular samples with approximate dimensions of 35.5×12×1.0 mm³ were isothermally tested at 25° C. and 1 Hz with a deflection of 15 μm by using single cantilever arrangement and the constant storage modulus values were recorded as the room temperature modulus (E′) of the cured sample. To determine the Tg and calculate crosslink density (ν) of the cured samples, additional rectangular DMA bars with approximate dimensions of 35.5×12.0×3.5 mm³ were scanned from −100° C. to well above their glass transition temperature at 1 Hz with a deflection of 15 μm while ramping the temperature with 5° C./min The temperature value of the maximum of the loss modulus peak was taken as the Tg value of the cured polymer and the (ν) values were calculated from the rubbery plateau of the temperature dependence of storage modulus curves at 50° C. above the Tg values by using Eq. 1.

E=3νRT (Eq. 1)

In Eq. 1, E is the storage modulus value of the sample at Tg+50° C. as determined via DMA and the T is the absolute temperature value 50° C. above the Tg of the material and R is the universal gas constant.

TGA measurements were performed to explore the thermal degradation profiles of the cured VE resins using a TA Instruments TGA (Q50). Tests were applied to the fully cured samples of all the compositions. Approximately 10-15 mg cured polymer was tested under argon atmosphere while thermally scanning the samples from 30 to 700° C. with a 10° C./min ramp. The weight loss as a function of temperature and rate of weight loss were measured. The Young's Modulus (E), tensile strength (σ), and failure strain (δ) of the VE formulations were determined via tensile tests in accordance with ASTM-D-638 standard test method using an Instron UTM (8800). Tensile tests were performed on dumb-bell-shaped (type IV) samples of all the VE formulations with different diluent concentrations. The nominal dimensions of the tensile bars were approximately 25 mm in height and approximately 2.0×2.0 mm² as measured from the neck of the tensile bars. Samples were tested at ambient conditions with the help of an extensiometer, using a constant crosshead speed of 1 mm/min and a gauge length of 45 mm. For each formulation, at least 6 tensile specimens were tested and analyzed.

### 3. Results and Discussion

### 3.1. Characterization of Vinyl Ester Monomers

The progress of the methacrylation reaction of the epoxy resins were also followed via acid number titrations by monitoring the amount of unreacted methacrylic acid in the mixture. Table 1 shows the result of acid number titrations for the methacrylation reaction for the different epoxy resins.

TABLE 1

| | Acid Number | | |
|---|---|---|---|
| Epoxy Resin | 1 h rxn time | 2 h rxn time | 3 h rxn time |
| DGEBA | 140 | 15 | 6 |
| SCECGE | 88 | 17 | 6 |
| NC514 | 52 | 21 | 7 |

As it can be seen from Table 1, 3 hours of reaction time was enough for all the epoxy resins to have an acid number value less than 10 which corresponds to a free acid amount of approximately 3 wt. %. The ¹H-NMR analyses along with the acid number titration results suggest that methacrylation of the epoxy resins were successful and almost all the methacrylic acid was reacted with the epoxide to form VERs. In addition, the time dependent acid number values showed that cardanol based resins and DGEBA epoxy had similar reactivity towards the methacrylic acid in the presence of AMC-2 catalyst.

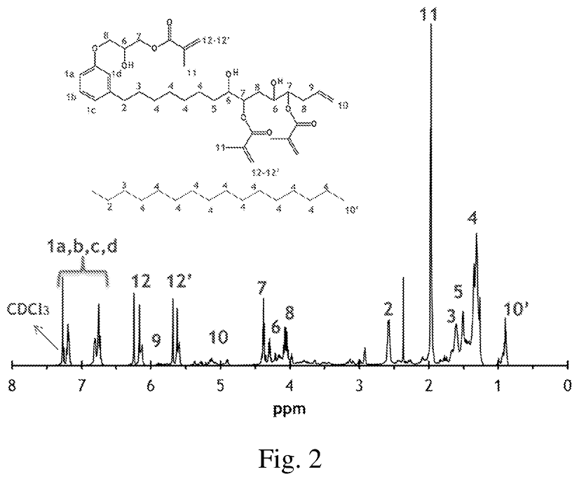

The ¹H-NMR spectra of the SCECGEVE is presented in FIG. 2 together with its molecular structure and corresponding peak assignments. Detailed ¹H-NMR analysis of the SCECGE reactant has also been explained previously. The addition of the methacrylate units was confirmed with the presence of the peaks at 6.25-5.65 ppm (12 and 12′) representing the methylene protons (CH═C(CH₃)) and the and the presence of the peak at 1.97 ppm (11) corresponding the methyl protons (—CH₃) of the methacrylate functional group. The multiplet peaks between 4.00-4.40 ppm (6, 7, and 8) were attributed to the (—OCH₂—) protons and tertiary —CH— protons (CH(OH)—) of the (phenyl-OCH₂—CH(OH)—CH₂O—(C═O)—) moiety respectively.

The average number of the methacrylate units in the NC514VE and DGEBAVE resins was calculated via the ¹H-NMR spectrum of the VE resins as explained by Jaillet, F.; Nouailhas, H.; Auvergne, R.; Ratsimihety, A.; Boutevin, B.; Caillol, S. European Journal of Lipid Science and Technology 2014, 116, (7), 928-939 and Can, E.; Kmaci, E.; Palmese, G. R. European Polymer Journal 2015, 72, 129-147 previously. The average number of methacrylate units in the SCECGEVE based resin was also calculated through a similar way via the integration ratio of the methylene protons of methacrylate end group at 6.15 and 5.6 ppm (12 and 12′), to the integration of the terminal methyl protons and methylene protons of the alkyl side chain at 0.88 ppm (10′) and at 5.1 ppm (10) respectively as internal standard peaks as shown in Eq.2. In Eq.2, Iₙ represents the integral values of the peaks with the designated numbers corresponding to different types of protons in structure presented in the ¹H-NMR spectra of SCECGEVE as indicated in FIG. 2. The constants 3 and 2 in Eq. 2 also represent the amount of the protons presented on the terminal methyl and terminal methylene protons of the side chain respectively.

$$F ( SCECGEVE ) - \frac{( {I}_{12} + {I}_{{12}^{′}} )}{( \frac{{I}_{10}}{3} + \frac{{I}_{{10}^{′}}}{2} )} \times ( \frac{1}{\begin{matrix}methylene protons of \\ the methacrylate ( 2 )\end{matrix}} ) \qquad ( Eq .2 )$$

The methacrylate functionality of the SCECGEVE macromere was calculated via eq. 2 and found as 2.4 methacrylate units per SCECGEVE molecule. The methacrylate functionalities of the NC514VE and DGEB AVE resins were calculated as it is explained by Jaillet and Can et al. by using their related ¹H-NMR spectra and found as ˜2.1 methacrylate units per NC514VE oligomer and 2.00 methacrylate units per DGEBAVE molecule respectively. The reason for the lower methacrylate content of NC514VE with respect to DGEBAVE was due to the oligomeric impurities which reduces the epoxy as well as methacrylate content of the resin. The ¹H-NMR analysis of DGEBAVE and NC514VE have previously been explored in detail. In addition, the molecular weight of the cross-linker units were calculated by their relative molecular structures and previously determined methacrylate functionality and determined as ˜540 g/mol for DGEBAVE and around 590 and 685 g/mol for SCECGEVE and NC514VE respectively.

### 3.2 Resin Viscosity

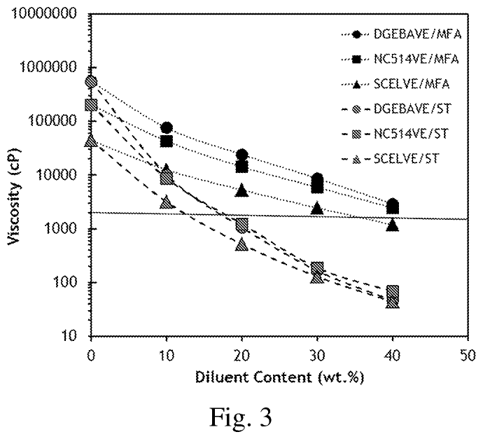

The viscosities of DGEBAVE/ST, DGEBAVE/MFA, NC514VE/ST, NC514VE/MFA, SCECGEVE/ST, and SCECGEVE/MFA mixtures with varying diluent content (10-40 wt. %) as well as the neat cross-linkers were determined at 25° C. and 20 s⁻¹ constant shear rate. The viscosity of the resin did not show a dependence on the shear rate and no shear history was observed meaning that all VE resins are Newtonian indicating no significant amount of polymerization occurred during resin preparation. The viscosity of the SCECGEVE resins as a function of reactive diluent content is presented in FIG. 3 along with that of NC514VE and DGEBAVE. Neat DGEBAVE and NC514VE resins showed significantly higher viscosities than that of the SCECGEVE resins for the same reactive diluent. The viscosity of the NC514VE was slightly lower than neat DGEBAVE despite its higher molecular weight because of the higher —OH content of DGEBAVE cross-linker molecule due to its better methacrylation efficiency. Also BPA core of the DGEBAVE may result in a higher viscosity for the DGEBAVE. Lowest viscosity among the neat VERs was observed for SCECGEVE because of its lower molecular weight with respect to NC514. Another reason can be the less bulky aromatic content of the SCECGEVE resin and the oligomeric impurities presented in the NC514VE which may result in increase in viscosity.

Addition of the bio-based MFA and synthetic ST reactive diluents to neat VEs dramatically reduced the viscosity of the blends. Neat MFA has a viscosity around 48 cP and styrene 0.7 cP. Hence, addition of ST reduced the viscosity of the resins to a significantly higher extent than MFA did, and hence the more negative slope for the styrenated resins in FIG. 3. ST is also a non-polar small molecule that behaves as a solvent, decreasing the intermolecular interaction among the larger and polar VE cross-linker monomer. On the other hand, MFA contains hydroxyl ester groups, which induce hydrogen bonding and increase the polar interactions thus reduces the viscosity of the VERs to a reduced degree. Typically the viscosity of the VERs should be between 500-2000 cP for most liquid molding resin applications. The viscosities of the all the VERs prepared with equal or more than 20 wt % styrene content and the SCECGEVE resin with 40 wt % MFA diluent were under 2000 cP.

### 3.3 Extent of Cure Analysis

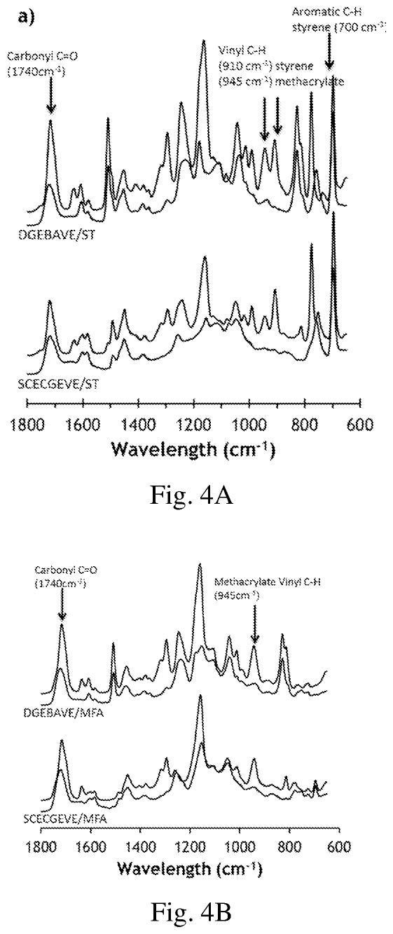

Upon thermal curing of the VE comonomer mixtures; hard, infusible polymeric materials were formed. FIGS. 4 (a) and (b) show the mid-IR spectra of DGEBAVE and SCECGEVE after post-cure in the presence of 30 wt % ST and MFA respectively along with their related uncured spectrum. The peak at 945 cm⁻¹; corresponding the stretch of vinyl bond of methacrylate group on the VE monomer was taken as the representative of reactive group, and the peak at 1750 cm⁻¹ which is the carbonyl peak was used as the internal reference. For the styrene conversion, the peak at 915 cm⁻¹ corresponding to stretch of vinyl bond was taken as the representative reactive group and the peak at 700cm⁻¹, indicative of the aromatic C═C stretch was used as the internal reference. The total conversion of VE was calculated via Eq. 3 and the conversion of styrene was determined using Eq. 4. The representative mid-IR spectra of the different VE blends containing 30 wt % diluent are presented in FIG. 4 for DGEBAVE and SCECGEVE cross-linker units.

$$\begin{matrix}{α}_{VE} = 1 - ( \frac{ABS ( t ) 945 {cm}^{- 1}}{ABS ( t = 0 ) 945 {cm}^{- 1}} ) ( \frac{ABS ( t = 0 ) 1750 {cm}^{- 1}}{ABS ( t ) 1750 {cm}^{- 1}} ) & ( Eq .3 ) \\ {α}_{ST} - 1 - ( \frac{ABS ( t ) 915 {cm}^{- 1}}{ABS ( t = 0 ) 915 {cm}^{- 1}} ) ( \frac{ABS ( t = 0 ) 700 {cm}^{- 1}}{ABS ( t ) 700 {cm}^{- 1}} ) & ( Eq .4 )\end{matrix}$$

The conversion of the VE and ST were calculated after post-cure are presented in Table 2. Table two is the extent of curing results after post-curing step for the different cross-linker units in the presence of 30 wt. % comonomer content.

TABLE 2

| | VE | ST | Overall |
|---|---|---|---|
| | Conversion | Conversion | Conversion |
| Sample | (%) | (%) | (%) |
| DGEBAVE/ST (70/30) | 84 ± 0.7 | 96 ± 1.0 | 92 ± 0.9 |
| SCECGEVE/ST (70/30) | 85 ± 1.1 | 95 ± 1.6 | 92 ± 1.4 |
| NC514VE/ST (70/30) | 81 ± 1.3 | 95 ± 1.2 | 91 ± 1.3 |
| DGEBAVE/MFA (70/30) | 89 ± 1.1 | | 89 ± 1.1 |
| SCECGEVEVE/MFA (70/30) | 86 ± 1.2 | | 86 ± 1.2 |
| NC514VE/MFA (70/30) | 85 ± 0.8 | | 85 ± 0.8 |

*Overall conversion for the styrenated formulations were calculated based on mole percentage.

The overall extent of cure for the styrenated formulations was also calculated basing on the mole fraction of the ST and VE and their corresponding individual extent of cure. Similarly, the overall methacrylate conversion including the VE and MFA was calculated via Eq.3 and presented in Table 2 as well. The overall conversion for the ST based formulations were higher than the MFA based ones because of the continuing polymerization of ST even after VE polymerization is done because of ST's high mobility in a gelled resin. Table 3 also showed that the different VE cross-linker units themselves have similar extent of cure in the presence of 30 wt. % ST or MFA diluents. Table 3 is a summary of the cured polymer properties for the different VE cross-linkers in the presence of 30 wt. % ST and MFA.

TABLE 3

| | Cross-link | | | | Tensile | Failure | | | |
|---|---|---|---|---|---|---|---|---|---|
| | Density (v) | Tg-LM | Tg-TD | E'(25° C.) | Modulus | Stress | Failure Strain | T_{10%} | Tₘₐₓ |
| VE Formulation | (mol/m³) | (° C.) | (° C.) | (° C.) | (E) (GPa) | (σ) (MPa) | (ϵ) (mm/mm) | (° C.) | (° C.) |
| DGEBAVE/ST 70/30 | 3250 ± 127 | 167 ± 2.2 | 176 ± 1.9 | 3.2 ± 0.3 | 3.3 ± 0.3 | 54 ± 3 | 0.012 ± 0.002 | 350 ± 3 | 450 ± 5 |
| SCECGEVE/ST 70/30 | 3750 ± 130 | 122 ± 2.1 | 135 ± 2.5 | 2.5 ± 0.3 | 2.6 ± 0.1 | 39 ± 2 | 0.016 ± 0.003 | 325 ± 2 | 395 ± 3 |
| NC514VE/ST 70/30 | 1500 ± 117 | 99 ± 2.2 | 113 ± 1.6 | 1.4 ± 0.3 | 1.1 ± 0.2 | 28 ± 2 | 0.1 ± 0.01 | 345 ± 4 | 425 ± 6 |
| DGEBAVE/MFA 70/30 | 7300 ± 113 | 132 ± 2.3 | 158 ± 2.1 | 3.0 ± 0.7 | 3.0 ± 0.3 | 39 ± 2 | 0.014 ± 0.002 | 334 ± 2 | 423 ± 2 |
| SCECGEVE/MFA 70/30 | 9800 ± 119 | 89 ± 1.9 | 123 ± 1.1 | 1.7 ± 0.3 | 1.6 ± 0.2 | 31 ± 1 | 0.061 ± 0.005 | 302 ± 1 | 395 ± 5 |
| NC514VE/MFA 70/30 | 2500 ± 115 | 63 ± 2.0 | 90 ± 1.6 | 0.35 ± 0.1 | 0.4 ± 0.05 | 24 ± 1 | 0.14 ± 0.01 | 327 ± 4 | 394 ± 3 |

Tg-LM: glass transition temperature as obtained from loss modulus maxima
Tg-TD: glass transition temperature as obtained from tan delta maxima

This extent of cure was above 80% suggesting that all the VE formulations cured to a high extent similar to that which is shown in the literature.

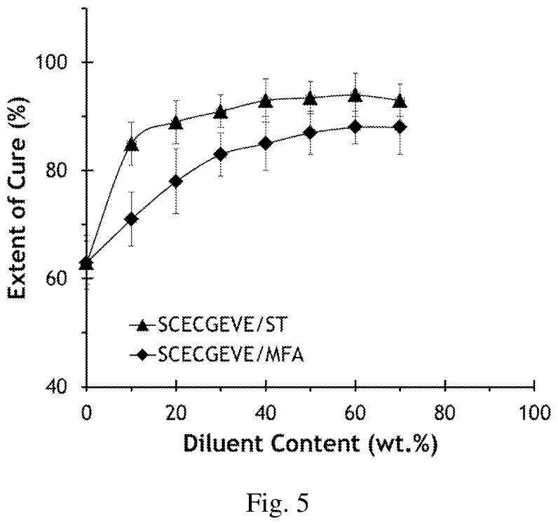

The effect of diluent content on the extent of cure of VERs was after post-cure and results are presented in FIG. 5. The neat SCECGEVE resin with no diluent had an extent of cure of 63%. Addition of MFA and ST comonomers progressively increased the extent of cure to 85% and 93%, respectively, because of reduced viscosity and the extent of cure at vitrification increases with diluent content. Yet, beyond 30 wt % diluent, the extent of cure does not increase any further suggesting that at least 30-40 wt % diluent content is required for SCECGEVE to achieve desired, high extent of cure.

### 3.4 Properties of the Cured VERs

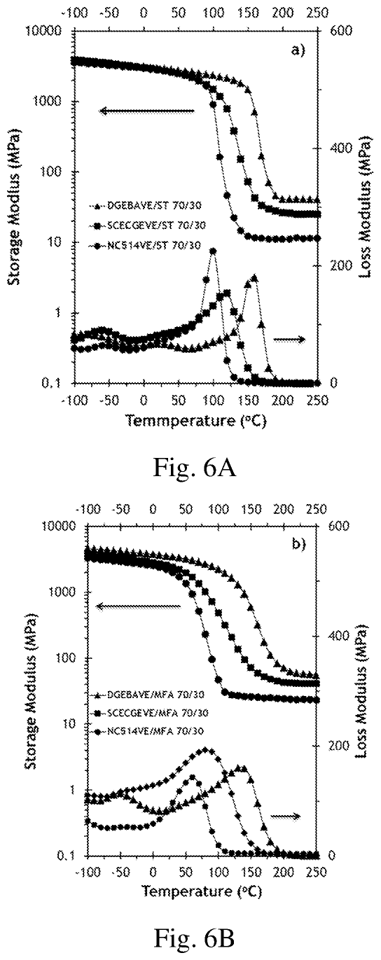

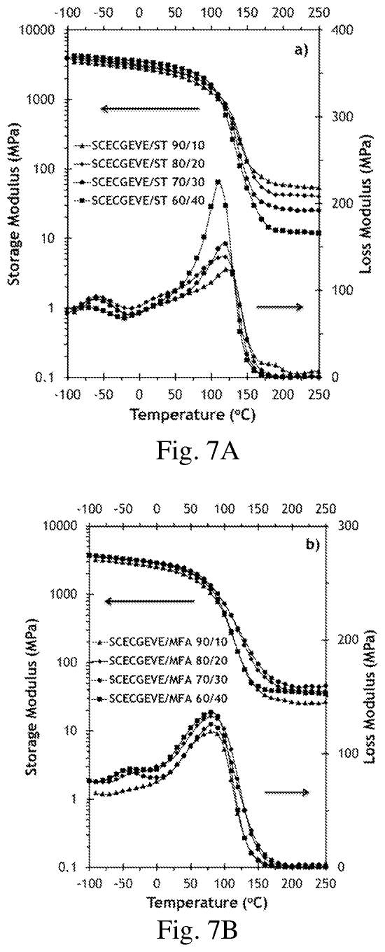

Thermo-mechanical properties of the fully cured VE formulations with different diluent contents were analyzed via DMA. FIGS. 6 (a) and (b) shows the temperature dependence of storage and loss modulus for DGEBAVE, SCECGEVE, and NC514VE cross-linkers prepared with 30 wt % ST and MFA comonomer. In addition, DMA results for SCECGEVE based formulations with varying comonomer content (10-40 wt %) and presented in the FIGS. 7 (a) and (b). The storage modulus is representative of the energy stored during cyclic deformation, and loss modulus is representative of energy that is dissipated as heat. The ratio of the loss modulus to the storage modulus is known as the tan delta (loss factor) and any of these properties can be used to predict the Tg of a material. The glass transition temperatures of the polymers were obtained via both temperature corresponding loss modulus maxima and tan delta maxima while the cross-link density values were obtained via the theory of rubber elasticity and reported in Table 3 and Table 4 for different cross-linker units and for SCECGVE with varying comonomer content, respectively. Table 4 is a summary of the cured polymer properties for SCECGEVE with changing comonomer content.

TABLE 4

| | Cross-link | | | | Tensile | Failure | Failure |
|---|---|---|---|---|---|---|---|
| | Density (v) | Tg-LM | Tg-TD | E'(25° C.) | Modulus (E) | Stress(σ) | Strain (ϵ) |
| VE Formulation | (mol/m³) | (° C.) | (° C.) | (GPa) | (GPa) | (MPa) | (mm/mm) |
| SCECGEVE/ST 90/10 | 7200 ± 143 | 125 ± 1.4 | 151 ± 1.9 | 2.7 ± 0.3 | 2.8 ± 0.4 | 48 ± 2 | 0.025 ± 0.008 |
| SCECGEVE/ST 80/20 | 6700 ± 153 | 123 ± 1.8 | 142 ± 1.5 | 2.6 ± 0.5 | 2.7 ± 0.3 | 43 ± 3 | 0.018 ± 0.007 |
| SCECGEVE/ST 70/30 | 3750 ± 130 | 122 ± 2.1 | 135 ± 2.5 | 2.5 ± 0.3 | 2.6 ± 0.1 | 39 ± 2 | 0.016 ± 0.002 |
| SCECGEVE/ST 60/40 | 2400 ± 13 | 117 ± 3.7 | 127 ± 2.1 | 2.4 ± 0.3 | 2.5 ± 0.4 | 26 ± 2 | 0.014 ± 0.003 |
| SCECGEVE/MFA 90/10 | 19250 ± 27 | 96 ± 1.4 | 145 ± 2.0 | 2.7 ± 0.7 | 2.4 ± 0.2 | 45 ± 1 | 0.042 ± 0.009 |
| SCECGEVE/MFA 80/20 | 13700 ± 38 | 91 ± 2.0 | 133 ± 1.8 | 2.3 ± 0.5 | 2.1 ± 0.5 | 40 ± 2 | 0.046 ± 0.004 |
| SCECGEVE/MFA 70/30 | 9800 ± 119 | 89 ± 1.9 | 123 ± 1.1 | 1.7 ± 0.3 | 1.6 ± 0.2 | 31 ± 3 | 0.061 ± 0.006 |
| SCECGEVE/MFA 60/40 | 6200 ± 38 | 85 ± 1.0 | 115 ± 1.7 | 1.3 ± 0.4 | 1.0 ± 0.1 | 17 ± 2 | 0.098 ± 0.009 |

Tg-LM: glass transition temperature as obtained from loss modulus maxima
Tg-TD: glass transition temperature as obtained from tan delta maxima

Among the cross-linker VERs, the highest cross-link density values were obtained for the SCECGEVE based formulations. DGEBAVE based formulations showed higher cross-link density values than the NC514VE based formulations. The highest cross-link density values observed for the SCECGEVE based formulations caused by the higher methacrylate content of the SCECGEVE monomer with respect to DGEBAVE and NC514VE. Furthermore, because of the oligomeric impurities presented in the NC514 epoxy resin which formed during the phenolation of cardanol, NC514VE has shown the lowest cross-link density values as a result of its lower methacrylate content. In addition, using MFA comonomer in VE formulations resulted in a higher cross-link density than using ST as commoner as shown in Table 3 because of the higher molecular weight of MFA which is around 3.3 times higher than the ST comonomer.

FIGS. 7 (a) and (b) shows that the loss modulus curve for 30 each of the VE formulations had a distinct peak attributed to its glass transition. Secondary transitions were not observed suggesting that the VE-diluent mixtures are completely miscible. FIG. 7 also showed that; DGEBAVE based VE formulations had the highest loss modulus Tg among the cross-linkers, which was around 157° C. and 132° C. in the presence of 30 wt % ST and MFA, respectively, because of its highly aromatic and bisphenol-A type of structure. On the other hand, despite the higher cross-link density of the SCECGEVE based formulations with respect to DGEBAVE based ones, SCECGEVE based formulations had lower Tg values around 122 and 89° C. (for ST and MFA, respectively) because of the presence of the C15 alkyd chain. NC514VE based formulations had the lowest Tg values among the cross-linkers which was around 99 and 69° C. for MFA and ST, respectively, despite its higher aromatic content with respect to SCECGEVE. The lowest Tg observed for NC514VE is because of its low methacrylate content which resulted in lower cross-link density. If NC514 and SCECGE had the same methacrylate content, the increased aromatic content of NC514 would be expected to raise the Tg relative to SCECGEVE, but the non-rigid ether linkage from the glycidyl functionality counteracts this effect as determined from group contribution theory. Additionally, VE formulations prepared with synthetic based ST showed higher glass transition temperature than the plant originated MFA based ones because of the rigid, aromatic nature of the styrene relative to the long aliphatic nature of the MFA which results in reduced Tg.

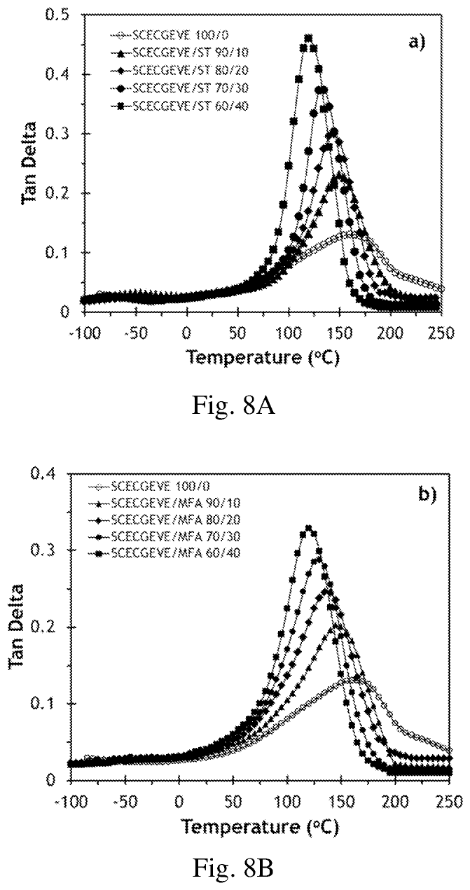

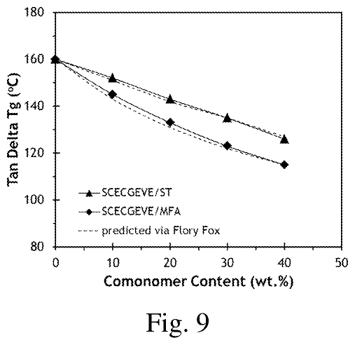

FIGS. 8 (a) and (b) shows the temperature dependence of storage and loss modulus as a function of comonomer content for SCECGEVE prepared with ST and MFA respectively. Because of the broad loss modulus peak, especially at lower diluent contents, a significant deviation was observed between the Tg values obtained via loss modulus DMA and predicted via Flory Fox's Method. Yet, the temperature for the maximum in tan delta did follow the Flory Fox Equation for these resins as shown in FIGS. 9 (a) and (b) and Table 4. Although the Tg values obtained via tan delta maxima is an over estimation, it is common for VERs to evaluate the Tg's via tan delta peak because this peak is sharper and with less variation between samples. Table 4 shows that addition of reactive diluents reduced the tan delta Tg of SCECGEVE dramatically because of the reduced cross-link density of the material with increasing comonomer content. A more pronounced reduction in Tg was observed in the presence of MFA with respect to ST as predicted. Others have demonstrated tan delta Tg agreement with the Flory Fox method³⁴⁻³⁶ It is important to note that, agreement with the Flory-Fox equation further verifies the miscibility of the VE-comonomer mixtures.

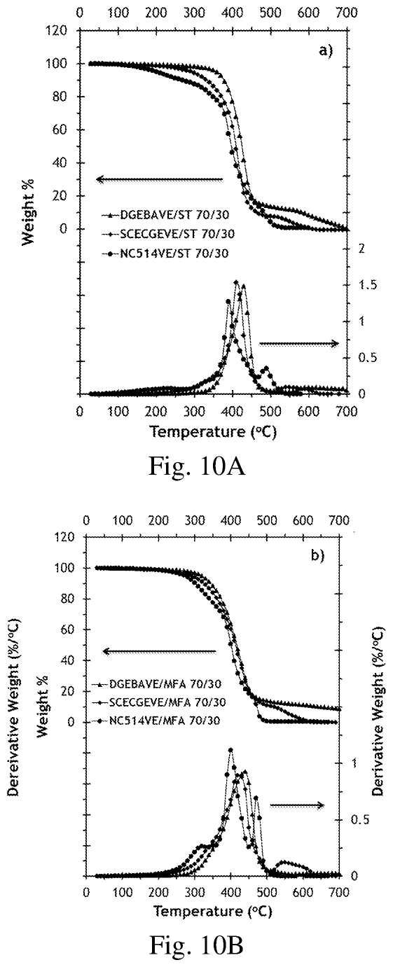

The thermal degradation profiles of the VERs evaluated under argon atmosphere via TGA. The thermo gravimetric curves of the VERs containing 30 wt % diluent was presented in FIG. 10. The temperature of 10% mass loss (T_{d-10%}) and the temperature of maximum degradation decomposition rate (T_{d-max}) were evaluated from the TGA traces and presented in Table 3 for the VE formulations prepared with 30 wt % diluent content. All of the cured samples showed thermal stability up to ˜240° C. and any weight loss started to happen above this temperature. The maximum rate of degradation was observed around 400° C., and after 600° C., almost complete volatilization of the samples occurred.

Among the cross-linker VE units, DGEBAVE based formulations had the highest T_{d-10%} because of its higher aromatic content with respect to SCECGEVE and higher cross-link density with respect to NC514VE. NC514VE based formulations had slightly higher thermal stabilities as a result of its increased aromatic content relative to SCECGEVE formulations. For example T_{d-10%} and T_{d-max} values for the DGEBAVE/MFA based formulations were 334 and 423° C. respectively. These values were reduced to 327 and 394° C. for the NC514/MFA based systems and to 312 and 395° C. for the SCECGEVE/MFA. Similarly, samples prepared with petroleum based styrene showed improved thermal properties than the plant based MFA. Presence of the C12 alkyd chain on the MFA molecule resulted in the reduction of the thermal stability since the long aliphatic chains degrade at significantly lower temperatures than the aromatic moieties. Indeed, as it seen from FIG. 10, NC514VE based formulations where the long aliphatic chain of cardanol is not cross-linked to a high degree, has shown degradation temperatures as low as 300° C. attributed to the degradation of the uncross-linked part of the C15 alkyd chain. This effect was more pronounced for the NC514VE/MFA formulations where uncross-linked alkyd chains presented from, both cross-linker and the diluent.

The tensile modulus (E) and the tensile strength (σ) of all the cured VE formulations were presented as a function of diluent content in Table 3 and 4 for different cross-linkers and for SCECGEVE and as a function of comonomer content, respectively. The E′ values obtained via isothermal DMA runs were also presented in Table 3 for different VE cross-linkers in the presence of 30 wt. % ST and MFA showed a good agreement with the Young's Modulus values obtained via tensile tests. Among the three cross-linker resins, the highest modulus (E) was observed for the samples prepared with the DGEBAVE cross-linker. DGEBAVE based VERs prepared with co-monomers had Young's Modulus values 3.3 and 3.0 GPa in the presence of 30 wt. % ST and MFA comonomer, respectively. VERs prepared with SCECGEVE cross-linker demonstrated lower modulus values of 2.6 and 1.6 GPa with ST and MFA diluents, respectively. VE formulations prepared with NC514VE based VE exhibited even more reduced E values with respect to DGEBAVE and SCECGEVE resins which were 1.1 and 0.4 GPa for the NC514VE prepared with ST and MFA, respectively. Similar trends were observed for the polymer strength (σ) with DGEBAVE>SCECGEVE>NC514VE. The reason for the highest modulus and strength values for the DGEBAVE based VE formulations were attributed to the highly aromatic nature and the bisphenol-A type rigid structure of the cross-linker unit. SCECGEVE based formulations, on the other hand, had lower values than the DGEBAVE based ones, in spite of the higher methacrylate functionality (2.4) and cross-link density because of the presence of the C15 alkyd chain on SCECGEVE molecule that reduces the stiffness and strength of the polymer. NC514VE based polymers exhibited the lowest modulus and strength values among the cross-linker units because of the low cross-link density caused by the lower methacrylate functionality relative to SCECGE. The Young's Modulus and strength of the SCECGEVE based formulations with increasing comonomer content presented in Table 4 showed that the modulus and strength decreased with the increasing comonomer content, likely due to the reduced cross-link density. However, the structure of co-monomer plays an important role as well. Increasing MFA content decreased modulus and strength to a higher extent than increasing ST content did due to the more flexible nature of the MFA co-monomer relative to the stiff, aromatic styrene comonomer.

Elongation at break trends were generally opposite to that of modulus and strength. The DGEBEVE had the lowest elongation at break, while the NC514VE had the highest. NC514VE had a higher elongation at break relative to SCECGEVE because of the lower crosslink density of NC514VE polymers. Yet, SCECGEVE had higher elongation at break than DGEBEVE because of the increased flexibility of the SCECGE polymer imparted from the alkyl chain despite its higher crosslink density than DGEBEVE polymers. Increasing styrene content in SCECGEVE resulted in reduction in elongation at break whereas increasing MFA content resulted in large increases in elongation at break. The MFA likely imparts additional flexibility to the network and reduces the crosslink density. The results for styrene were unexpected as generally decreasing the crosslink density should make the have greater flexibility and increased conversion up to 30 wt % styrene should result in at least improved elongation to break up to 30 wt % styrene.

### 4. Conclusions

A novel low viscosity cardanol based VER was synthesized from SCECGE via reaction with methacrylic acid. The curing studies of the different VERs in the presence of varying contents of reactive diluent showed that SCECGEVE cured to similar extents as the other VERs and increasing reactive diluent improved conversion up to 30 wt % reactive diluent. Thermo-mechanical properties obtained via DMA measurements showed that SCECGEVE based formulations had significantly improved loss modulus and tan delta Tg values in the presence of ST and MFA diluents (122-89° C. as obtained from loss modulus) relative to NC514VE (99-63° C.) because of the higher methacrylate functionality for SCECGEVE. The Young's Modulus and strength of the VE formulations were also higher for SCECGEVE relative to that of NC514VE. Despite the low volatility, the high viscosity and the presence of fatty acid backbone on MFA molecule reduced the processability and polymer properties with respect to ST molecule and would require 30 wt %+diluent content for most composites liquid processing applications. Thus, these diluents should be used as the ternary blends to adjust the most suitable viscosity and the optimum cured polymer properties. SCECGEVE resin also had significantly lower viscosity than it is cardanol based counterpart NC514VE and synthetic based counterpart DGEBAVE. Overall, resin and polymer properties showed that SCECGEVE is a significantly better than NC514VE resin for composites and coatings applications. However, cured polymer properties of SCECGEVE were lower than the DGBEVE based formulations indicating that these Cardanol-based resins would be useful for moderate temperature applications and not the higher temperature applications more applicable to DGEBAVE resins. Finally, the SCECGEVE molecule has shown significantly improved Tg and E values in VE form with respect to SCECGE-PACM epoxy-amine system because of the complete reaction of the functional groups which resulted in a highly cross-linked network and significantly improved Tg and Young's Modulus.

Similar results are expected to be attainable by the use of various different methods to fully or partially (meth)acrylate the cardanol molecule.

As those skilled in the art will appreciate, numerous modifications and variations of the present invention are possible in light of these teachings, and all such are contemplated hereby. For example, in addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes, or at least for the purpose described in the context in which the reference was presented.

### References

The following references may be useful in understanding some of the principles discussed herein:

1. Agrawal, S.; Mishra, A.; Rai, J. Journal of applied polymer science 2003, 87, (12), 1948-1951.

2. Sultania, M.; Rai, J.; Srivastava, D. European Polymer Journal 2010, 46, (10), 2019-2032.

3. Can, E.; Kinaci, E.; Palmese, G. R. European Polymer Journal 2015, 72, 129-147.

4. Sultania, M.; Rai, J. S. P.; Srivastava, D. Journal of Hazardous Materials 2011, 185, (2-3), 1198-1204.

5. Garg, M. S.; Srivastava, D. Progress in Organic Coatings 2014, 77, (7), 1208-1220.

6. Shelby, M. Ntp Cerhr Mon 2008, (22), v, vii-ix, 1-64 passim.

7. Legislatures, N. C. o. S. NCSL POLICY UPDATE: STATE RESTRICTIONS ON BISPHENOL A (BPA) IN CONSUMER PRODUCTS. http://www.ncsl.org/research/environment-and-natural-resources/policy-update-on-state-restrictions-on-bisphenol-a.aspx

8. Khot, S. N.; Lascala, J. J.; Can, E.; Morye, S. S.; Williams, G. I.; Palmese, G. R.; Kusefoglu, S. H.; Wool, R. P. Journal of Applied Polymer Science 2001, 82, (3), 703-723.

9. Can, E.; La Scala, J.; Sands, J.; Palmese, G. Journal of applied polymer science 2007, 106, (6), 3833-3842.

10. LaScala, J. J.; Jeyarajasingam, A.; Winston, C.; Sand, J. M.; Palmese, G. R. Predicting the Viscosity of Low VOC Vinyl Ester and Fatty Acid-Based Resins; DTIC Document: 2005.

11. La Scala, J. J.; Sands, J. M.; Orlicki, J. A.; Robinette, E. J.; Palmese, G. R. Polymer 2004, 45, (22), 7729-7737.

12. Bassett, A. W.; Rogers, D. P.; Sadler, J. M.; La Scala, J. J.; Wool, R. P.; Stanzione, J. F. Journal of Applied Polymer Science 2016, 133, (45).

13. Jaillet, F.; Nouailhas, H.; Boutevin, B.; Caillol, S. European Journal of Lipid Science and Technology 2016.

14. Auvergne, R.; Caillol, S.; David, G.; Boutevin, B.; Pascault, J.-P. Chemical Reviews 2013, 114, (2), 1082-1115.

15. Campanella, A.; Scala, J. J. L.; Wool, R. Journal of Applied Polymer Science 2011, 119, (2), 1000-1010.

16. Voirin, C.; Caillol, S.; Sadavarte, N. V.; Tawade, B. V.; Boutevin, B.; Wadgaonkar, P. P. Polymer Chemistry 2014, 5, (9), 3142-3162.

17. Tyman, J.; Wilczynski, D.; Kashani, M. Journal of the American Oil Chemists' Society 1978, 55, (9), 663-668.

18. Dinesh Balgude, A. S. S. Journal of Coatings Technology & Research 2014, 11, (2), 14.

19. Verge, P.; Toniazzo, V.; Ruch, D.; Bomfim, J. A. Industrial Crops and Products 2014, 55, 180-186.

20. Sultania, M.; Rai, J.; Srivastava, D. Materials Chemistry and Physics 2012, 132, (1), 180-186.

21. Sultania, M.; Yadaw, S.; Rai, J.; Srivastava, D. Materials Science and Engineering: A 2010, 527, (18), 4560-4570.

22. Sultania, M.; Rai, J.; Srivastava, D. Journal of applied polymer science 2010, 118, (4), 1979-1989.

23. Jaillet, F.; Nouailhas, H.; Auvergne, R.; Ratsimihety, A.; Boutevin, B.; Caillol, S. European Journal of Lipid Science and Technology 2014, 116, (7), 928-939.

24. McAninch, I. M.; Palmese, G. R.; Lenhart, J. L.; La Scala, J. J. Polymer Engineering & Science 2015, 55, (12), 2761-2774.

25. Fouquet, T.; Puchot, L.; Verge, P.; Bomfim, J. A.; Ruch, D. Analytica chimica acta 2014, 843, 46-58.

26. Jaillet, F.; Darroman, E.; Ratsimihety, A.; Auvergne, R.; Boutevin, B.; Caillol, S. European Journal of Lipid Science and Technology 2014, 116, (1), 63-73.

27. Ziaee, S.; Palmese, G. R. Journal of Polymer Science Part B: Polymer Physics 1999, 37, (7), 725-744.

28. La Scala, J. J.; Orlicki, J. A.; Winston, C.; Robinette, E. J.; Sands, J. M.; Palmese, G. R. Polymer 2005, 46, (9), 2908-2921.

29. Brill, R. P.; Palmese, G. R. Journal of applied polymer science 2000, 76, (10), 1572-1582.

30. Robinette, E.; Ziaee, S.; Palmese, G. Polymer 2004, 45, (18), 6143-6154.

31. Rekha, N.; Asha, S. Journal of applied polymer science 2008, 109, (5), 2781-2790.

32. Sadler, J. M.; Toulan, F. R.; Nguyen, A.-P. T.; Kayea, R. V.; Ziaee, S.; Palmese, G. R.; La Scala, J. J. Carbohydrate polymers 2014, 100, 97-106.

33. Van Krevelen, D. W.; Te Nijenhuis, K., Properties of polymers: their correlation with chemical structure; their numerical estimation and prediction from additive group contributions. Elsevier: 2009.

34. Fox Jr, T. G.; Flory, P. J. Journal of Applied Physics 1950, 21, (6), 581-591.

35. Flory, P. J.; Rehner Jr, J. The Journal of Chemical Physics 1943, 11, (11), 512-520.

36. Flory, P. J., Principles of polymer chemistry. Cornell University Press: 1953.

1. A polymerizable monomer prepared by partial or complete acrylation or methacrylation of at least one epoxidized cardanol, wherein the epoxidized cardanol is formed by epoxidation of an unsaturation site of a cardanol having the formula: 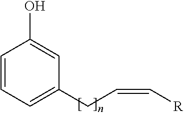
wherein R is selected from hydrogen and an alkyl, or alkenyl group having 1-6 carbon atoms, and n is 7, 10, or 13.

2. The polymerizable monomer of claim 1, wherein said epoxidized cardanol is cardanol glycidyl ether.

3. The polymerizable monomer of claim 1, prepared by reacting (meth)acrylic acid with the at least one epoxidized cardanol in the presence of a metal-containing catalyst.

4. The polymerizable monomer of claim 1, prepared by reacting (meth)acrylic anhydride with the at least one epoxidized cardanol.

5. The polymerizable monomer of claim 1, prepared by reacting (meth)acryloyl chloride with the at least one epoxidized cardanol in the presence of one or or more of triethylamine, tertiary amines, pyridines and pyridine derivatives.

6. The polymerizable monomer of claim 3, wherein the at least one epoxidized cardanol has from about 2.0 to about 4.0 epoxy groups per molecule.

7. The polymerizable monomer of claim 3, wherein the at least one epoxidized cardanol has from about 2.2 to about 3.0 epoxy groups per molecule.

8. The polymerizable monomer of claim 1, wherein the (meth)acrylation is carried out in the presence of an effective amount of a free radical polymerization inhibitor.

9. The polymerizable monomer of claim 1, wherein the (meth)acrylation is carried out in the presence of an effective amount of a catalyst for carboxylic acid-epoxy resin systems, said catalyst being selected from of amines, phosphines, antimony compounds, and imidazoles.

10. The polymerizable monomer of claim 1, wherein the (meth)acrylation is carried out using a molar ratio of (meth)acrylate groups to epoxide groups in the epoxidized cardanol of from about 0.5 to about 1.1.

11. A polymerizable monomer prepared by partial or complete acrylation or methacrylation of at least one hydroxylated cardanol, wherein the hydroxylated cardanol is formed by hydroxylation of at least one unsaturation site of a cardanol having the formula: 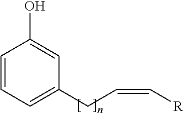
wherein R is selected from hydrogen, and an alkyl group, or alkenyl goup having 1-6 carbon atoms, and n is 7, 10, or 13.

12. The polymerizable monomer of claim 11, prepared by reacting (meth)acrylic anhydride with the at least one hydroxylated cardanol.

13. The polymerizable monomer of claim 11, prepared by reacting (meth)acryloyl chloride with the at least one hydroxylated cardanol in the presence of triethylaluminium.

14. The polymerizable monomer of claim 11, wherein the (meth)acrylation is carried out using a molar ratio of (meth)acrylate groups to hydroxy groups in the hydroxylated cardanol of from about 0.5 to about 1.1.

15. A polymerizable monomer comprising a vinyl ester of the formula: 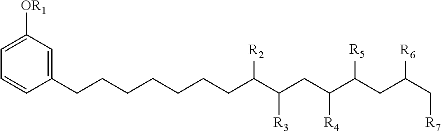
wherein R₁ is selected from one of the following, hydrogen, propylene oxide, 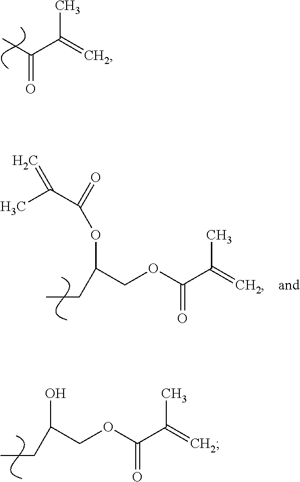
and
R₂, R₃, R₄, R₅, R₆ and R₇ are independently selected from hydrogen, hydroxyl, an acrylate group, a methacrylate group, and an epoxy groups formed by combinations of R₂ and R₃, R₄ and R₅, and R₆ and R₇; and wherein at least one of R₂, R₃, R₄, R₅, R₆ and R₇ includes an acrylate group or a methacrylate group.

16. A method of making a polymer comprising the step of curing a composition comprising the polymerizable monomer of claim 1.

17. The method of claim 16, wherein the composition further comprises from 1 to 99% by weight of at least one reactive diluent, based on the total weight of the composition.

18. The method of claim 17, wherein the reactive diluent is selected from the group consisting of: styrene, 2-hydroxymethacrylate, methyl methacrylate, methyl acrylate, furfuryl methacrylate, methacrylated lauric acid, methacrylated C₆-C₂₄ fatty acids, methacrylated guaiacol, methacrylated phenol, and methacrylated eugenol.

19. The method of claim 17, wherein the reactive diluent is a methacrylated C₆-C₂₄ fatty acid.

20. The method of claim 17, wherein said composition comprises from about 50% to about 90% by weight of the polymerizable monomer of any one of claims 1-14 and from about 10% to about 50% by weight of the reactive diluent, based on a total weight of the composition.

21. The method of claim 17, wherein the composition comprises from about 30% to about 50% by weight of said reactive diluent.

22. The method of claim 17, wherein the curing step is carried out in the presence of an effective amount of a polymerization initiator.

23. A polymer obtained by the method of claim 17.

24. The polymer of claim 23 having a Tg of from about 0° C. to about 125° C., wherein the Tg is the temperature at which the largest peak of the loss modulus occurs, as measured by Dynamic Mechanical Analysis method IOP L1047-033 with the following parameters: test frequency=1 Hz, strain=0.02%, and heating rate of 2° C./min, a modulus of more than 1 GPa at 25° C. and a strength of more than 10 MPa at 25° C.

25. The polymer of claim 23, having a Tg of from about 100° C. to about 150° C. wherein the Tg is the temperature at which the largest peak of the loss modulus occurs, as measured by Dynamic Mechanical Analysis method IOP L1047-033 with the following parameters: test frequency=1 Hz, strain=0.02%, and heating rate of 2° C./min.

26. A resin comprising the polymerizable monomer of claim 1 and a reactive diluent, wherein the resin has a viscosity of from about 500 cP to about 2000 cP.
